(19) United States
(12) Patent Application Publication
(10) Pub. No.: US 2024/0038352 A1
(43) Pub. Date: Feb. 1, 2024

(54) METHOD AND APPARATUS FOR PRESCRIPTION MANAGEMENT

(71) Applicants: Shafaat Pirani, Lutz, FL (US); Brett Thompson, Lutz, FL (US)

(72) Inventors: Shafaat Pirani, Lutz, FL (US); Brett Thompson, Lutz, FL (US)

(21) Appl. No.: 18/373,195

(22) Filed: Sep. 26, 2023

Related U.S. Application Data
(60) Provisional application No. 63/392,121, filed on Jul. 26, 2022.

(51) Int. Cl. G16H 20/10; G06Q 20/36; G06Q 20/42; G06Q 20/40; G06N 3/092

(52) U.S. Cl. CPC G16H 20/10; G06Q 20/36; G06Q 20/42; G06Q 20/40; G06N 3/092

(57) ABSTRACT

The present system is a cloud-based prescription management service that can be used by patients, providers, pharmacies, payors, and pharmaceutical manufacturers to assist with the coordination of prescription management. The system can be accessed via any processing device, such as a smartphone, tablet, desktop, and the like. A patient retains autonomy in selecting where the prescription is ultimately dispensed and can track all existing prescriptions, manage prescriptions, review the status of prescriptions, establish refill reminders, and compare insurance coverage and pricing preferences. The system allows a user to choose one or more payment methods, manage multiple users, delivery preferences, and delivery locations for medications. The system acts as a hub that is integrated with a network of pharmacies where that prescription is “transferred” electronically from the system Digital Wallet or application to the pharmacy of choice.

This patent application claims priority to the U.S. Provisional Application 63/392,121 filed on Jul. 26, 2022 which is incorporated by reference herein in its entirety.

### BACKGROUND OF THE SYSTEM

The filling of prescriptions has become a complex process. A patient must coordinate with their provider, pharmacy, payor, and the pharmacy benefit management (PBM) company to ensure that the prescription is covered by the patient's insurance. In addition, the patient must choose a pharmacy that accepts the patient's insurance and within their PBM's network. There are also several price comparison and price reduction applications that can be used to determine the lowest price for the prescribed medication (e.g., GoodRx, SingleCare, Pharmacy Checker, Amazon Pharmacy, Rx Saver, and the like). If the patient does use insurance, the claim must be handled properly, and coverage must be reviewed to make sure it is correct and that the proper amounts have been paid and/or credited to a patient deductible. There are many different coverage plans, and the coverage varies with each plan. In addition, there are circumstances where it may be better to not use insurance and pay out-of-pocket for a prescription. However, it is difficult to make all of these calculations and decisions at the moment of purchase or fulfillment of a prescription.

Furthermore, patients receive a plethora of calls from both the pharmacy and the provider's office whilst coordinating their prescriptions, family members, and for those when they are listed as a caretaker. From the collection of co-pays, to providing updated demographics and insurance information, possible changes from a brand to a generic (where substitution is permitted), to arranging pick-up, delivery, and or mailed options. For all these reasons and more, this convoluted process creates an undue burden resulting in complexity, confusion, expense, and delays in initiating therapy for all the participants in a prescription transaction.

### SUMMARY

The present system is a cloud-based prescription management service that can be used by patients, providers, pharmacies, payors, and pharmaceutical manufacturers to assist with the coordination of prescription management. The system can be accessed via any processing device, such as a smartphone, tablet, desktop, and the like. A patient retains autonomy in selecting where the prescription is ultimately dispensed and can track all existing prescriptions, manage prescriptions, review the status of prescriptions, establish refill reminders, and compare insurance coverage and pricing preferences. The system maintains a complete audit of each prescription so that the full history is always available per state and federal regulations. The system allows a user to choose one or more payment methods, manage multiple users, delivery preferences, and delivery locations of medications. The system can engage through texts, emails, push notifications, manual entry, and the like. The user may opt to allow updates to inform the user of the progress of the prescription transaction. When problems arise, the user can act on them immediately and or engage with the customer support team to promptly resolve.

The system acts as a hub that is integrated with a network of pharmacies where that prescription is “transferred” electronically from the system Digital Wallet or application to the pharmacy of choice. The system architecture is designed methodically with several layers of integration across multiple partners. These integrations include a pharmacy's management software (PMS) system, a pharmacy's point-of-sale (POS) system, multiple national delivery logistics partners, a national payment processing partner, and other ancillary partners that create an integrated, interoperable, and holistic solution with robust clinical utility.

### BRIEF DESCRIPTION OF THE DRAWINGS

FIG. 1 is a flow diagram of the operation of the system in an embodiment.

FIG. 2 is a flow diagram illustrating pharmacy choice in an embodiment of the system.

FIG. 3 illustrates an interface of a System App in an embodiment.

FIG. 4 illustrates an interface of the System App showing an example of a prescribed medication in an embodiment.

FIG. 5 illustrates an interface of the System App showing a system Digital Wallet in an embodiment.

FIG. 6 illustrates prescription history and details of a medication in an embodiment.

FIG. 7 illustrates a timeline in an embodiment.

FIG. 8 is a block diagram of the architecture of the system in an embodiment.

FIG. 9 illustrates an example of neural network training in an embodiment.

FIG. 10 is an example computer system for implementing the system in an embodiment.

### DETAILED DESCRIPTION OF THE SYSTEM

The system provides an end-to-end prescription management solution that operates under a SaaS-based technology (Software as a Service) that is responsible for the transmission of information from one software solution to another. In one embodiment, the platform is a patient/consumer information vehicle that transmits prescription information between the provider's electronic health record (EHR), the patient, and the pharmacy's prescription management system (PMS). Each of the components services acts as an information reservoir that is interconnected to the centralized middleware. Delivery partners act as third-party logistic partners that pick up a package (prescription) from a pharmacy and deliver it to a requested destination. No core medical and or prescription information regarding the actual patient is disclosed.

The system transmits prescription orders to a patient's pharmacy of choice for all non-control medications per state and federal guidelines. The system provides patients with a solution that is consumer-centric for both cash and insurance-based medications. This includes the collection of co-pays, assessing delivery fees, customizing tips for the driver, applying discount savings via prescription discount cards for cash prescriptions, and or the use of manufacturer co-pay cards for commercial payors. In one embodiment, the system will prompt a user regarding any insurance co-pay requirement and will make the determination of state vs. federal plans (e.g., Centers for Medicare and Medicaid Services, Tricare, etc.) to ensure manufacturer co-pay cards are not applied to federal claims. The system provides 100% passthrough for regulatory compliance in one embodiment. In one embodiment, the delivery partner will not be authorized to deliver the prescription until payment has been collected. In one embodiment the delivery partner will not be privy to patient medical information including but not limited to DOB, insurance plan, prescription drug name, other prescription characteristics, and or diagnosis/medical condition. This ensures that both the system and the delivery partner are not in violation of any HIPAA, anti-kickback statutes, Stark law, patient steering laws, and or any action that could be misconstrued as inducing and or incentivizing a consumer to fill a medication funded by a federal program.

System Operation

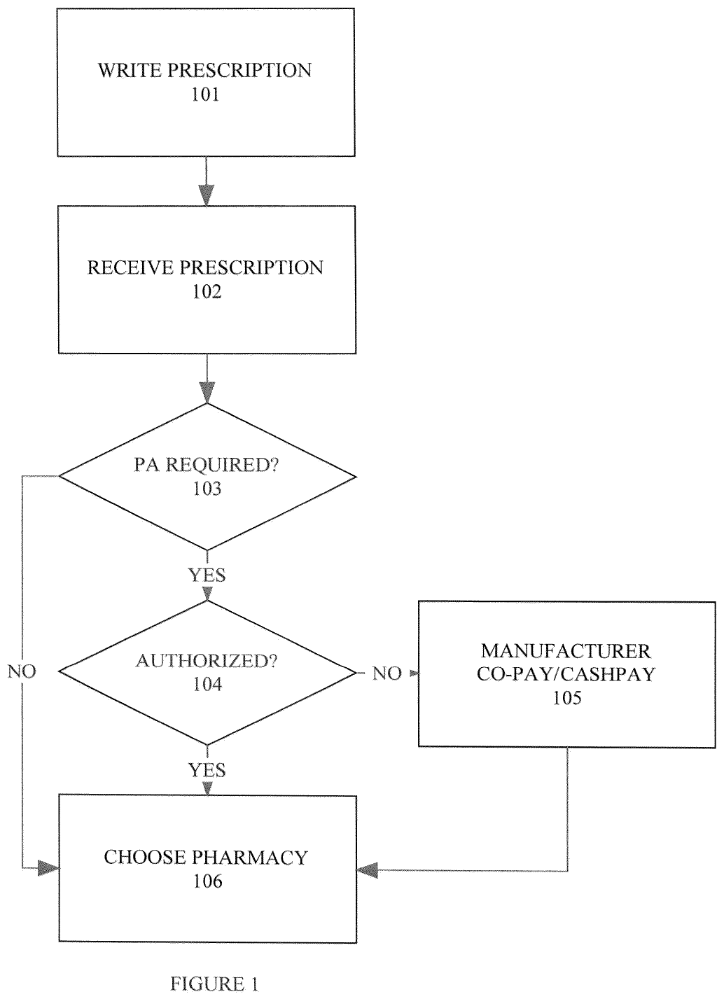

FIG. 1 is a flow diagram of the operation of the initial steps of the system in an embodiment. At step 101 a healthcare professional writes a prescription for the user. The Healthcare professional in one embodiment uses an electronic health record software that integrates with the SureScripts networks. The prescription is electronic and can be transmitted easily to any suitable pharmacy. The prescribing healthcare professional selects the system, which is powered by an independent pharmacy. The technology platform utilizes a series of advanced programming interfaces (APIs) to expose the prescription in the patient apparatus of choice (e.g., mobile application, tablet, desktop) as it is tied to the mobile phone number of the patient. If the patient's first name, last name, mobile phone number, and DOB are an exact 1:1 match, the patient will be able to view the prescription and go about managing their prescription thus providing a digitized experience. If the fields are not a 1:1 exact match, the system will provide a set of challenges for the user to unlock the prescription, thus, providing an extra layer of security with the appropriate technical safeguards to prevent the unintended or accidental exposure of personal health information (PHI), which would violate HIPAA. The successful receipt of the prescription at step 102 enables management of the prescription by the user within the system. The platform has unique functionality allowing for a master profile to control profiles created for users under the age of eighteen and to enable prescription management for those above of the age of eighteen when the appropriate HIPAA consents are obtained. This enables the entire household to be managed under one profile as is common in the U.S. household.

At decision block 103 it is determined if prior authorization is required for the prescription. Some insurance plans require prior authorization before allowing the user to fill the prescription. If prior authorization may be required, the system places the prescription on hold and informs the user. Decision block 104 depicts the background prior authorization processes occurring. Based on the type of insurance involved and the outcome, the prescription may be eligible for manufacturer co-pay assistance which is auto-applied when consent is obtained or a cash-program may be offered depending on the manufacturer. Prior authorization services may be implemented by a Prescription Drug Authorization Service partner, which can interact with the user's insurance company and health provider to provide prior authorization information or it can be handled internally by the pharmacy support staff.

In one embodiment, the prior authorization analysis is implemented using an Artificial Intelligence (AI) system that uses conditional logic, processes, data resolution, and injection to perform tasks including determining if prior authorization is needed, if there are FDA controls that require rejection of the prescription such as the medication being a controlled substance, and or if an accountable care organization (ACO) provider or manufacturer has certain clinical requirements that necessitate the prescription be held or routed to a specific in-network pharmacy to complete dispensing. These examples include patient enrollment in a care program for routine monitoring, a limited distribution network based on drug supply, requiring a specialty pharmacy to dispense the prescription, and others as applicable.

If authorization is denied at step 104, the system proceeds to step 105 to determine if there are cash-pay programs offered by the pharmaceutical manufacturer or prescription drug discount cards (i.e., SingleCare, GoodRx, etc.) available for the medicine and or other alternatives that can be used to assist with affordability. The use of cash-pay programs and or prescription drug discount cards can help promote medication adherence when there are barriers to filling the medication but are often too difficult to navigate or the patient may be unaware of these options. The system uses AI and machine learning to determine eligibility, cost advantages, compliance, opportunities for therapeutic interchange, and the like.

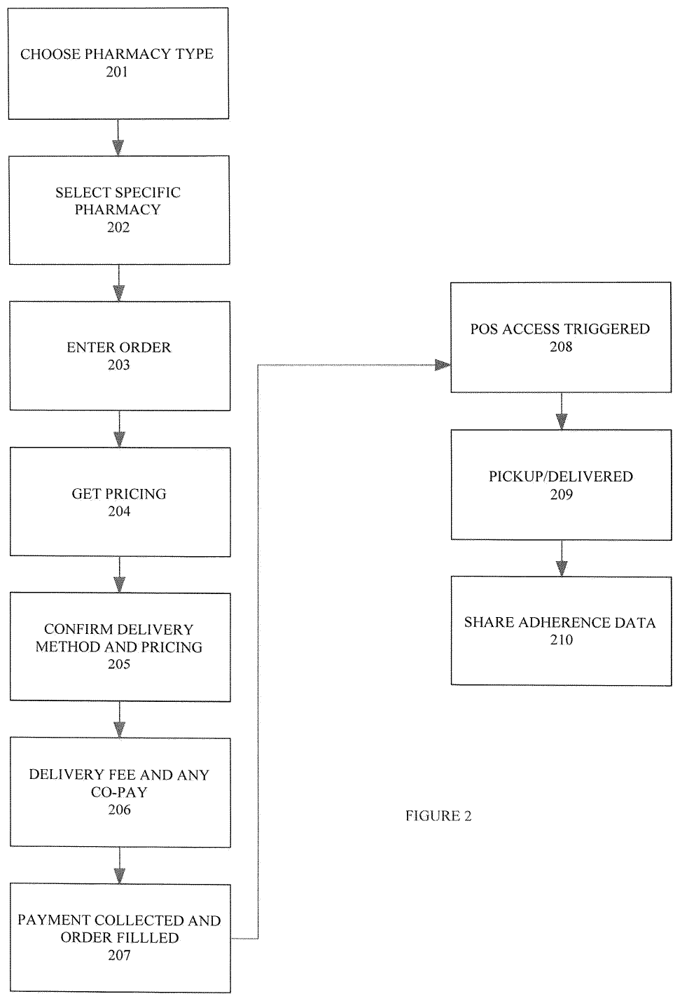

After step 105, or if the authorization is granted at step 104, or if no authorization is required at step 103, the system will use AI and machine learning to review the insurance coverage and apply pharmaceutical manufacturer co-pay cards when eligible. Manufacturers of drugs will sometimes offer special promotions to increase awareness and/or use of the medication. These offers when eligible can include partial payment of the co-pay to bring down the out-of-pocket costs to the patient which assists with access and affordability. The system provides information to the user about these offers and promotions to help defray the cost of the prescription. The manufacturer co-pay programs are specific to certain drug NDCs (e.g., SKUs) which can be complex and are subject to change. The system uses AI and machine learning to determine eligibility, cost advantages, compliance, therapeutic interchange, and the like. Upon conclusion of this robust process, the patient proceeds to choose a pharmacy at step 106, which is described in the flow diagram of FIG. 2.

At step 201, the user selects their preferred method of prescription receipt which includes the traditional pick-up option, same-day or next-day delivery, or have their medication shipped to the requested address. The system uses AI and machine learning to cross-examine patient preference, patient geo-location, the specific medication, and various pharmacy criteria to display eligible pharmacies capable of servicing the prescription(s) in a timely fashion. In one embodiment of the system, these eligible pharmacies are comprised of three different types of networks: the integrated network and receives the prescription electronically, the soft network of pharmacies which is defined as a network partner pharmacy that is not integrated with the platform and receives the prescription via facsimile transmission, or a general pharmacy not part of the integrated or soft network of partner pharmacies and also receives it via facsimile transmission. If no prior authorization is required at step 103, the user can choose any of the three types of pharmacies. If prior authorization is required at step 104, the user can select either the integrated network or soft network of pharmacies due to the backend coordination that needs to be completed to fill the medication therapy per the pharmaceutical manufacturer or provider-based organization. If prior authorization is not granted at step 104 and the user takes advantage of a cash-based program or drug discount card, then they are able to select any of the three types of pharmacy networks which is usually based on price and prescription turnaround time.

Once the user has chosen a type of pharmacy at step 201 based on the AI filtering and eligibility criteria set forth, the user can then filter pharmacies based on preference and select the specific dispensing pharmacy at step 202. The system will also provide prompts and analysis for allergy check, interaction with other known medications of the user, use of AI for provision of insurance information, and the like before transferring the prescription to the pharmacy of choice. This may include automatic checking for allergy issues based on the ingredients of the medication and recorded allergies of the user that are already in the system. It may also include direct queries to the user regarding allergy information. In one embodiment, the system may ask the patient to confirm their allergies in a dialog box on the user app. Thereafter, the prescription is transferred electronically or via facsimile based on the type of pharmacy. At step 203, the pharmacy has already received the prescription and has begun entering it into their system which triggers webhooks into the core platform technology which is relayed to the patient based on their communication preferences (e.g., SMS, email, and or push notifications within the mobile application). These communications are streamlined, therefore, only events requiring action by the patient are filtered through. If the patient would like to see a full account of their prescription processing history, they can access this information in the events timeline within the mobile application. Note, in one embodiment, if a soft network partner pharmacy or general pharmacy is chosen, the patient will coordinate with the pharmacy for payment and delivery. All soft network partner pharmacies will export adherence data in our proprietary system.

At step 204, if an integrated network partner pharmacy is chosen, the patient will begin to receive the relevant webhooks back into their mobile application which includes the total price of the medication, specifically the covered insurance amount and co-pay, as applicable, and other miscellaneous fees depending on the method of receipt.

At step 205 the user chooses and confirms a receipt method (e.g., pick-up, courier delivery, or shipping) and confirms the pricing. The user is presented with the price for the receiving method originally selected. The user can then agree to the estimated price and confirm their original receipt method choice, or the user can change to pick-up if desired. The system manages the delivery fee payment and any co-pay requirement at step 206 through its integration with Stripe and use of the multi-party system API. This API enables the system to collect all relevant fees from the patient or user and then disseminate them appropriately to the applicable parties (i.e., the system and the dispensing pharmacy). Co-pays are 100% transmitted to the pharmacy and the fees that the dispensing pharmacy pays the system are pulled after the deposit is made for compliance purposes. This multi-party API completes all the transactions seamlessly and within seconds providing an accurate account for the flow of funds. Overall, the system supports cash or insurance pricing, collects payment, and distributes the payment to the stakeholders in the system.

At step 207 the payment for the order is collected via the mechanism explained above and ultimately the order is filled. The Point-of-Sale (POS) APIs are triggered at step 208. The POS API is called once payment has been collected to create a notification message to the dispensing pharmacy that payment has already been collected and the amount due upon checkout is zero dollars. The core platform utilizes this API call to instruct the pharmacy to have the order ready for pick-up by the patient, courier delivery, or shipping along with the corresponding order ID number. Other notes are passed through this API including whether the patient needs to be counselled on the prescription therapy to satisfy the requirements of OBRA 90. If delivery is selected by the patient, a pharmacy staff member performs a function within the POS which serves as a trigger to call the API for courier delivery with one of the integration partners mentioned below. At step 209 the prescription is picked-up, delivered, or shipped depending on the method chosen by the user. At step 210, adherence data is shared to the system by the participants either through automatic electronic means if it is an integrated network partner pharmacy or through a soft network partner pharmacy via data drop portal. This data is analyzed by the core to facilitate adherence related reports which in turn are de-identified and externalized with end-client users such as pharmaceutical manufacturers or provider-based organizations. The core platform technology has the ability to facilitate bi-directional communications from either the user or the dispensing pharmacy to conduct pharmacy processes. This includes functions such as refill reminders which can be initiated by other party when appropriate, and communicated to the other party to either facilitate processing or accepting the order.

In one embodiment, the delivery of the prescription may be accomplished by a rideshare system such as Lyft, delivery systems such as Roadie, or package carriers such as UPS, USPS, FedEx, and the like). In one embodiment, payment processing may be implemented using Stripe, Pay with Extend, and the like. Process partners in one embodiment can include, SureScripts, BestRx, Digital Business Solutions, PrimeRx, PioneerRx, and the like. Prescription drug discount card partners include Paramount Rx, SingleCare, and the like.

User App

In one embodiment, a user (e.g., a patient, or person receiving a prescription) uses a System App to manage their prescriptions. The System App may be implemented on an application on a mobile device, desktop, tablet, and the like. The user will choose the system as their desired pharmacy with their health care providers which will enable the prescription to flow into the System Platform and be exposed on the device of their choice.

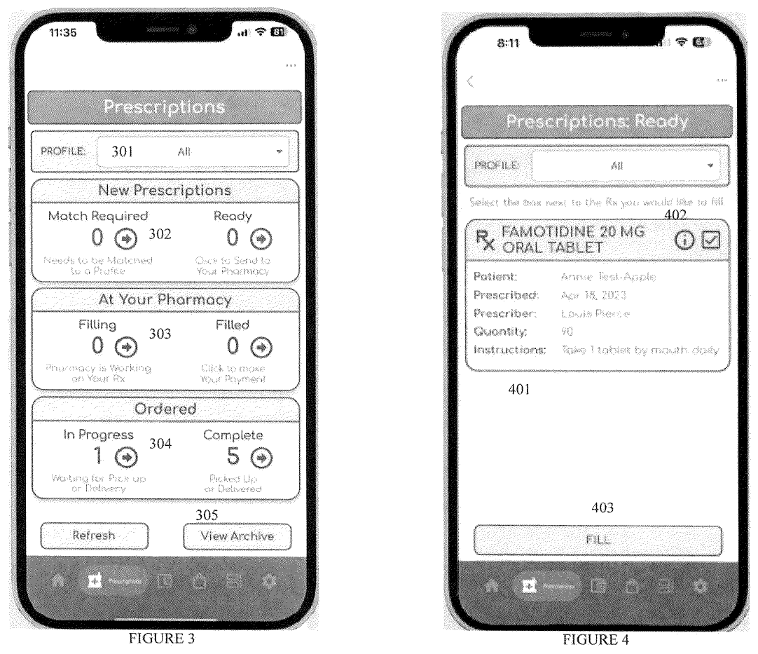

FIG. 3 illustrates an interface of the System App implanted on a smartphone. The interface includes a region 301 where a user profile can be selected and viewed or all profiles under the master account. Region 302 shows new prescriptions, if any. In some cases, different providers may have different names for the user. In that case, the System App may require help in matching a prescription with a user. In one embodiment, the system uses AI and/or machine learning to help identify the user and may require the user to confirm ownership of the prescription before proceeding. Region 302 also shows the number of prescriptions that are ready and can be send to the user's pharmacy by clicking on an arrow or button and following the prompts based on the choices that the user selects.

Region 303 indicates prescriptions at that have been transferred to the pharmacy that are either being filled or filled. A button or arrow allows the user to make a payment for those prescriptions that are filled. Region 304 illustrates those prescriptions that have been ordered in region 303 and shows the number in progress on the left and the number that are complete on the right. An Archive button 305 can be selected to view historical prescription data as desired. Data is automatically archived if an order has been marked as complete for a set amount of time to avoid confusing or inundating the user a large number of prescriptions on the home screen of the app.

FIG. 4 illustrates an interface of the System App showing an example of a prescribed medication in an embodiment. Region 401 shows the medication prescribed (e.g., Famotidine), the strength (e.g., 20 MG), the quantity, patient/prescriber information, and directions for use information. An information button “i” 402 can be selected to provide more information about the medication including the prescription timeline, a drug monograph, picture of the medication, and the like. The drug monograph information is dynamic, therefore if the dispensing pharmacy utilizes a different manufacturer for a generic medication, the drug monograph image will be automatically updated with the exact color and shape of the medication that is ultimately dispensed. The user must select the check box to the right of medication card and then choose to fill the prescription by activating Fill button 403. This enables users to fill one or several medications at any given time.

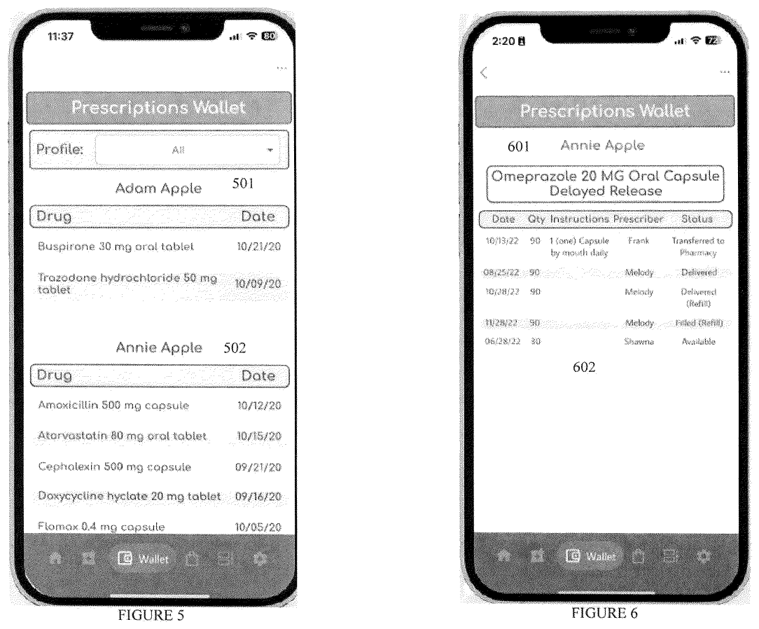

FIG. 5 illustrates an interface of the System App showing a system Digital Wallet in an embodiment. This interface includes a region 501 for a first user “Adam Apple” and a region 502 for a second user “Annie Apple.” For each user their medications are listed with date of most recent transaction included. Selecting one of the medications results in the interface of FIG. 6, illustrating prescription history and details of that medication. Region 601 shows the user and the name of the medication. Region 602 provides a history of each prescription filled, the date, and quantity prescribed, as well as the status for each entry.

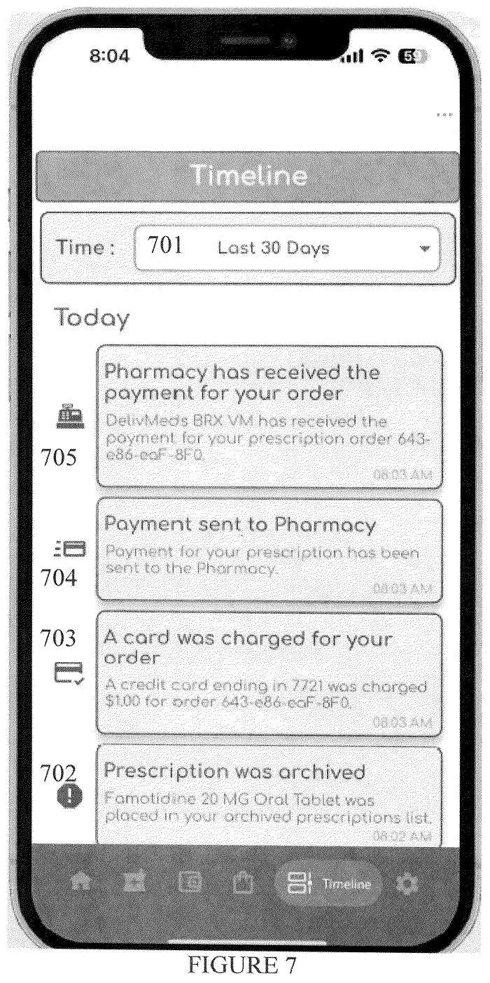

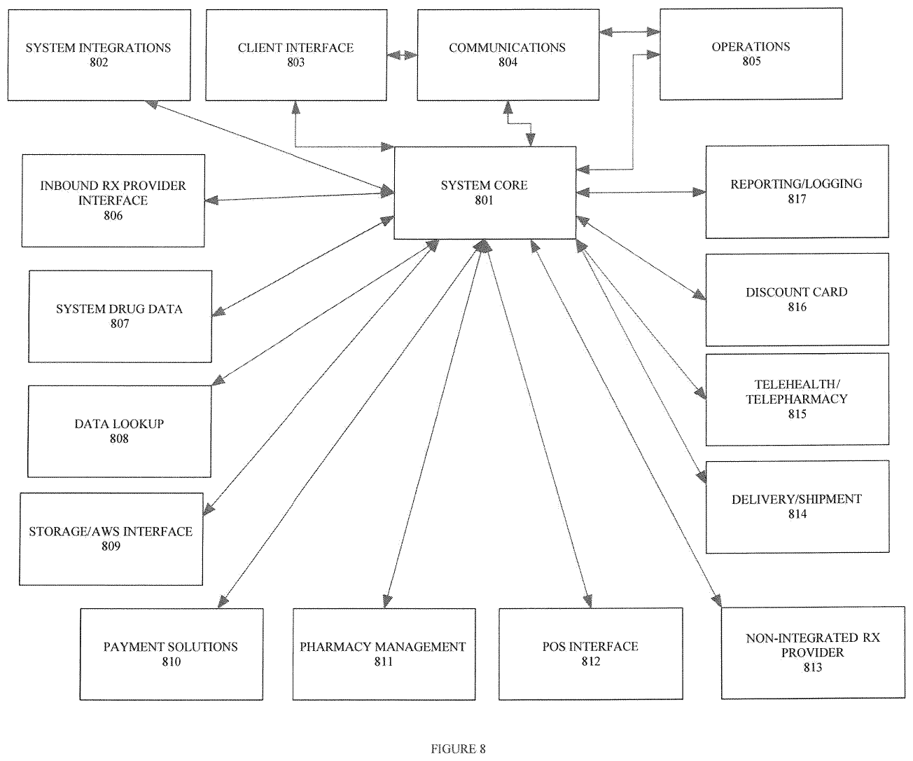

In one embodiment, the System App provides a Timeline function that allows the user to see the flow of events related to a prescription. Region 701 allows the user to select a time frame of events (e.g., last 30 days). In one embodiment, the timeline is arranged with newest entry on top and oldest on the bottom. In the example of FIG. 7, message 702 states that the prescription was archived in the user's prescription list. Message 703 states that a credit card was charged for the order.

Message 704 shows that payment was sent to the pharmacy chosen by the user and message 705 shows the confirmation that the pharmacy has received the payment. The System App allows a user to refill a prescription directly from the app. In addition, the System App accesses databases to provide information about medications, side effects, possible interactions with other medications, and the like. The system offers the user the opportunity to use multiple pharmacies selected by the user as well as a host of pickup and delivery options. The system may check for allergies known to be associated with the user. This analysis may be performed in one embodiment by AI and/or machine learning.

The system integrates directly with the pharmacy management software systems to allow additional information exchange between the system and the Pharmacies. The core architecture is mapped to pharmacy workflow with pre-defined triggers that enable events to fire autonomously, no additional action is needed from the pharmacy staff. Additionally, to avoid potential compromises with personal health information (PHI), the system generates an Order ID and additional challenge token that is exposed to the delivery individuals, therefore, when presenting to a partner pharmacy they do not need to have the date of birth (DOB) or any other personal information of the user.

The system allows for completing the payment transaction prior to pick-up, delivery, or shipping. Using the System App, the user can provide patient consent through the app and share the consent directly with the pharmacy system. The user is required to acknowledge the co-pay amount, confirm the method of receipt, and either accept or decline pharmacist counselling services for the prescription(s) being filled. This information is collected with a simple dialogue box where the user selects yes or no and provides their digital signature which can be completed with their finger. This information is “pushed” into the dispensing pharmacy's PMS and POS systems. The System App provides the ability to add additional language, instructions, etc., on the screen to the pharmacist with instructions (e.g., this will be picked up by delivery driver Joe, please verify via this ID and challenge token); or instructions to use Delivery Partner approved packaging (opaque, tamper evident packaging with no PHI printed anywhere on it) and other useful and custom messages and instructions.

The System App and all platforms connecting to the core, maintains the gold-standard of pharmaceutical regulatory compliance at both the state and federal level. If there is a particular type of information exchange, consent, record keeping practice that can help provide compliance and/or risk mitigation that can be accomplished with these integration and partnership capabilities, the system is able to put those components in place to achieve that compliance and/or handle that risk mitigation. Further, the System App does not allow for control medications (CII-CV) to be transferred through the hub network through its various integrations and APIs with the Food & Drug Administration (FDA) to cross-check federal databases to confirm. Various state boards of pharmacies are reviewed continuously to ensure there are no additional products that are added to these databases which may lead to a compliance violation.

The System App, and the drug information, timelines, system digital wallet, orders, and the like, are implemented in one embodiment by AI and/or machine learning.

System Architecture

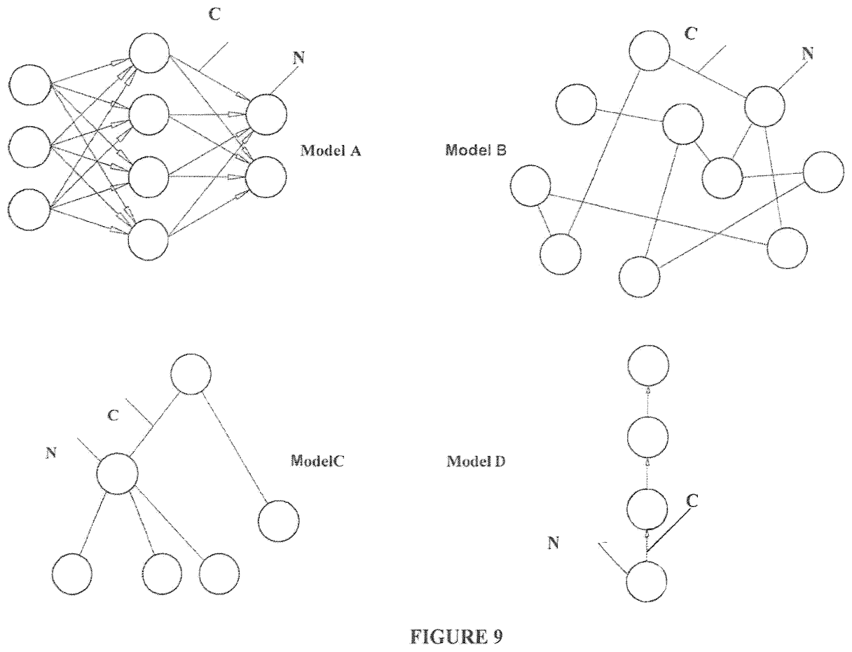

FIG. 8 illustrates the System Architecture in an embodiment. There are multiple platforms that are part of the solution herein. The system core 801 manages the entire system and interfaces with the different platforms. The system core 801 utilizes AI and/or machine learning to help provide intelligent routing of prescriptions. The system core provide general services, user account management, patient prescription management, orders (including an order system for prescriptions, delivery fee support, smart shipping logic, transfers, refills, and the like), smart services, additional services, and pharmacy logic.

Pharmacy logic includes pharmacy lookup for the type of pharmacy, prescription receipt method for a user, geo-location, tracking pharmacy hours, open and closed days, holiday schedule, pharmacy state licensing status which would enable more dispensing pharmacy options for a user if shipping is selected, payor/PBM contracts and associated restrictions, pharmacy's clinical service capability, fax services, and pharmacy data collection services. In one embodiment, pharmacy logic is implemented using AI and/or machine learning. This logic is crucial to the overall hub operation and practicality of the application as it streamlines the transfer of the prescription by intelligently routing the prescription to a pharmacy capable of servicing the patient based on prescription characteristics and patient preference.

System integration interface 802 provides a way for future external partners interact with our system as we have published robust API endpoints and webhooks for the system.

The Client Interface 803 provides APIs to interface with the System App on a user device (iOS, Android, and the like), a web client portal for manufacturers and provider groups and a separate pharmacist portal for soft network partner pharmacies to update pharmacy information and share data. The manufacturers receive adherence reporting to allow them to do more targeted marketing. The providers receive information about patient outcomes and PDC (prescription days covered) which is directly correlated with adherence data. This interface 803 also controls an Admin portal for pharmacists, technicians, customer service representatives, developers, executives, and the like to review data in aggregate and perform certain tasks related to the system.

Communications interface 804 is used to handle messaging in the system between users of the application and the core system. Many of these communications are automated and or based on event types which trigger notifications. The following external partners are used to facilitate these communications with patients: SendGrid, Twilio, Firebase, iOS push notifications, and Android push notifications. Operations integrations interface 805 controls internal systems, communications with team members or automation when a critical issue is discovered and a message is transmitted to the development team (e.g., Slack), customer service, ticketing system (e.g., Zoho) to allow the system to field and troubleshoot various consumer raised issues.

The inbound prescription provider interface 806 allows interaction with the primary prescription intake provider BestRx (or any other prescription intake provider). In one embodiment, the integrated network of partner pharmacies using the Best Rx system are able to electronically receive e-transfer prescriptions from our core platform technology. In one embodiment, prescriptions submitted by a physician or other health provider are entered into the electronic health record and transmitted over the SureScripts network which is then received by the pharmacy via the inbound BestRx PMS, which communicates with the System Core 801, and eventually into the System App for users to view, transfer, and manage.

The system supports integration with various Pharmacy Management System (“PMS”) Rx and POS systems. The system is scalable and can partner with any number of Rx and POS systems. In one embodiment, the system handles a standardized format to support all the features, and then there are providers for each partner that are developed to communicate to their APIs if they have them, and if they do not, the system can provide a plugin the partner can install in their system to talk to the system core. In one embodiment, the system can normalize PMS/POS data so that the system can work with that partner.

System Drug Data interface 807 provides communication with system drug information storage, including drug monographs, drug names, drug data, images, and the like. This interface can also communicate with third party resources for drug information, including, in an embodiment, the BestRx drug database. In one embodiment, the system can also engage with prospective partners such as VUCA Health, Elsevier, and the like for video information related to drugs, medications, how to administer, and the like.

Data lookup interface 808 provides communication with other drug information resources including the FDA drug database for compliance related checks (e.g., controlled drugs) and it provides geo-location services to provide additional compliance checks on location of users of the system.

Storage/AWS interface 809 provides an interface to servers on a system such as Amazon Web Services in an embodiment.

Payment solutions interface 810 is used to communicate with and engage payment processing partners. In one embodiment, the system uses a service such as Stripe, and various APIs provided and supported by Stripe including Multi-Party Tenant API and virtual credit card webhooks, Pay with Extend, and the like.

Pharmacy management interface 811 integrates pharmacy partners for transferring a prescription to the service provider pharmacy that will actually fill the prescription for the user. In one embodiment, the system uses the BestRx PMS transfer API, Refill API, prescription updates webhooks, and patient messaging. The system may use other or multiple PMS providers as desired, such as PrimeRx, PioneerRx, Digital Business Solutions, and the like.

POS interface 812 provides system integration into the POS systems used by the partner pharmacies. The BestRx POS API is used to communicate messages from the System Core to the partner pharmacies. Future pharmacy integrations will dictate the need of subsequent POS integrations to drive the solutions end-to-end capability.

Non-integrated Rx provider interface 813 allows the system to interact even with pharmacies that are not currently integrated into the system, but that a user may desire to select for filling of a prescription. Because these pharmacies are not integrated for digital communication, the system is able to provide fax transmission of prescriptions to the pharmacy of choice, however, they are unable to collect payment, schedule delivery, and unable to facilitate bi-directional communication. Therefore, alternate systems have been put into place to facilitate additional services with soft network partner pharmacies.

Delivery/Shipment interface 814 is used to communicate with and manage shipping and courier delivery of filled prescriptions. Shipping delivery includes UPS, USPS, and FedEx by leveraging an integration with EasyPost. Courier delivery is powered by Lyft and Roadie via native integrations with those partners directly. In one embodiment, the System Core leverages APIs with the PMS and POS systems and that of the courier or shipping provider to facilitate communications and the transfer of information such as notifications, status updates, estimated time of delivery, driver information, tracking numbers, and the like. A patient may prefer delivery but is not sure which pharmacy provides that service, or in what range. The system will review pharmacies near the GPS coordinates of the user and find a suitable pharmacy that will deliver to that location. If desired, the user can choose to pick-up the prescription.

Telehealth/Tele-pharmacy interface 815 provides users with the ability to connect with a third-party partner to utilize telehealth services in the event that a prescription is needed. Similarly, the mobile application enables for users to connect with a virtual pharmacist via synchronous video connection or asynchronously in the form of chat-based messaging.

Discount card interface 816 is for cash-based transactions when insurance benefits generate large out-of-pocket co-pays for covered medications and use of a prescription drug discount card can provide large savings on the out-of-pocket costs for said medication. In one embodiment SingleCare and Paramount Rx have both been integrated into the Core System and when applicable and with patient consent, can be auto-applied to inform the user of the expected out-of-pocket costs for a medication through cash-pay vs. the insurance price.

Reporting/logging interface 817 is used to provide audit trails of all events and transactions in the system. In one embodiment a service called Sentry is used to track and log errors/issues regarding the system and can be used to improve performance. The reporting feature enables the System Core to aggregate data and return results in the form of reports that are de-identified and externalized with end-client partners such as the pharmaceutical manufacturers and provider-based organizations.

AI/Machine Learning

Because of the large number of partners, providers, and participants in the prior authorization process, writing, filling, payment, and ultimate delivery of prescriptions to an end user, the ability to train an AI or to implement machine learning has been very difficult, if not impossible. The present system helps overcome that problem in at least two ways. In one embodiment, the system normalizes input data from different sources to allow more efficient ingestion of the data. For example, different manufacturers may use different terms, fields, and even field order in describing their products, while still complying with federal disclosure regulations. The present system can normalize drug data from different manufacturers and PMS systems to make it easier to ingest data by the AI.

In one embodiment, the system access data that would not be available to individual partners. By defining the system as a preferred pharmacy by a user, the prescription becomes available to the system in digital form, improving the ability to apply AI and machine learning to the prescription and the fulfilment process.

Further, by implementing tokens for each user that do not compromise personal health information (PHI), the system allows for more robust analysis without violating HIPPA restrictions, adding to the efficiency of an AI system. The system utilizes a reinforcement model for learning that is routinely monitored and evaluated. Due to the high number of prescription claims moving through the system, there are a plethora of opportunities to continue evaluating the learning for process improvement. A loss function is calculated during the training period and routine tweaks and adjustments are made to improve the learning and system reliability.

The reinforcement learning (RL) model is a type of machine learning approach used in artificial intelligence (AI) training. It is inspired by behavioral psychology and focuses on training agents to make sequential decisions in an environment to maximize a cumulative reward signal. In one embodiment, a reinforcement learning model utilizes Agents, Environment, States, Action, Policy, Reward, Trajectory, Value Function Exploration, Learning Algorithm, and Markov Decision Process (MDP).

Agent: The agent is the AI entity that interacts with its environment and makes decisions. It can be a robot, processing node, or any other system designed to learn and perform tasks.

Environment: The environment is the external system with which the agent interacts. It encompasses everything that can affect the agent and be influenced by its actions. Environments can be real-world, simulated, or abstract, depending on the application.

State (s): A state represents the current situation or configuration of the environment. It contains all the relevant information needed for the agent to make decisions. States can be discrete or continuous, depending on the problem.

Action (a): An action is a decision or choice made by the agent at each time step. The set of possible actions an agent can take is called the action space.

Policy (π): The policy is the strategy or function that defines how the agent selects actions based on the current state. It is an important component of RL and is what the agent learns to optimize over time.

Reward (r): A reward is a numerical signal provided by the environment to the agent after it takes an action in a particular state. The reward indicates the immediate desirability or quality of the action taken. The goal of the agent is to maximize the cumulative reward over time.

Trajectory or Episode: A trajectory or episode is a sequence of states, actions, and rewards that the agent experiences while interacting with the environment. RL algorithms use these trajectories to learn and update the policy.

Value Function (V or Q): The value function estimates the expected cumulative reward that an agent can achieve from a given state (V) or a state-action pair (Q). It is an important component in many RL algorithms, helping the agent evaluate and compare different states or actions.

Exploration vs. Exploitation: One of the challenges in RL is the trade-off between exploration and exploitation. The agent must explore new actions and states to discover the best policy while exploiting its current knowledge to maximize short-term rewards.

Learning Algorithm: RL employs various learning algorithms to train the agent's policy or value function. Common RL algorithms include Q-learning, Deep Q-Networks (DQN), Policy Gradient methods, and actor-critic methods.

Markov Decision Process (MDP): RL problems are often formalized as Markov Decision Processes, where the Markov property assumes that the future state depends only on the current state and action, making it a memoryless process.

The RL training process typically involves the agent interacting with the environment, collecting data, and using this data to update its policy or value function iteratively. Through this process, the agent learns to make better decisions to maximize its cumulative reward over time.

Reinforcement learning has been successfully applied in various domains, including robotics, game playing, autonomous vehicles, recommendation systems, and healthcare, among others. It is a powerful approach for training AI systems to learn and adapt to complex, dynamic environments.

In RL, a loss function is a mathematical expression that quantifies how well an agent is performing a given task or how far off its predictions are from the desired outcomes. The loss function plays a crucial role in the training process as it guides the agent's learning by providing a measure of the error or discrepancy between the agent's actions or predictions and the optimal or expected behavior.

The primary purpose of the loss function in RL is to help the agent adjust its policy (strategy) or value function (approximation of expected rewards) so that it can improve its decision-making and maximize cumulative rewards over time. Examples of loss functions include Policy Loss, Value Loss, and Actor-Critic Loss.

Policy Loss: In policy-based methods, the loss function is designed to optimize the agent's policy. The policy loss typically quantifies how well the agent's actions align with the expected or desired actions that lead to higher rewards. The choice of policy loss depends on the specific RL algorithm being used. In one embodiment, a Policy Gradient Loss may be used. This loss function is often used in policy gradient methods like REINFORCE. It encourages actions that have resulted in higher rewards to have higher probabilities under the policy.

Value Loss: In value-based methods, the loss function is used to estimate the error in the agent's value function (either state-value or action-value). The value loss measures the discrepancy between the predicted values and the actual rewards observed during training. In one embodiment, a Mean Squared Error (MSE) loss function may be used. This loss function is frequently used in value-based methods such as Q-learning and Deep Q-Networks (DQN). It minimizes the squared difference between the predicted Q-values and the target Q-values, which are typically computed using the Bellman equation.

Actor-Critic Loss: Actor-critic methods combine elements of both policy and value-based approaches. They have separate networks for the policy (actor) and the value function (critic). The loss function often includes components related to both the policy and the value function, helping the agent simultaneously improve its action selection and value estimation.

The choice of the specific loss function depends on the RL algorithm, the problem at hand, and the design of the agent's neural network or learning model. The agent's objective is to minimize this loss during training by adjusting its policy or value function parameters through techniques like gradient descent. As the agent learns and iteratively improves its policy, the loss function ideally decreases, indicating better performance and more effective decision-making.

The loss function serves as an important component in the RL training process, providing a quantitative measure of how well the agent is progressing toward its goal of maximizing cumulative rewards.

Referring to FIG. 9, the teaching presented by the disclosure can be implemented to include various other artificial intelligence models (“AIMs”) and/or techniques. The disclosed devices, systems, and methods for learning and using AIMs are independent of the artificial intelligence model and/or technique used and any model and/or technique can be utilized to facilitate the functionalities described herein. Examples of these models and/or techniques include deep learning, supervised learning, unsupervised learning, neural networks (i.e., convolutional neural network, recurrent neural network, deep neural network, etc.), search-based, logic and/or fuzzy logic-based, optimization-based, tree/graph/other data structure-based, hierarchical, symbolic and/or sub-symbolic, evolutionary, genetic, multi-agent, deterministic, probabilistic, statistical, and/or other models and/or techniques.

In one example shown in Model A, the disclosed devices, systems, and methods for learning and using AIMs may include a neural network (also referred to as artificial neural network, etc.). As such, machine learning, knowledge representation or structure, pattern recognition, decision making, and/or other artificial intelligence functionalities may include a network of Nodes N (also referred to as neurons in the context of neural networks, etc.) and Connections C similar to that of a brain. Node N can store any data, object, data structure, and/or other item, or reference thereto. Node N may also include a function for transforming or manipulating any data, object, data structure, and/or other item. Examples of such transformation functions include mathematical functions (i.e. addition, subtraction, multiplication, division, sin, cos, log, derivative, integral, etc.), object manipulation functions (i.e. creating an object, modifying an object, deleting an object, appending objects, etc.), data structure manipulation functions (i.e. creating a data structure, modifying a data structure, deleting a data structure, creating a data field, modifying a data field, deleting a data field, etc.), and/or other transformation functions. Connection C can store or be associated with a value such as a symbolic label or numeric attribute (i.e., weight, cost, capacity, length, etc.). A neural network can be utilized as a predictive modelling approach in machine learning. A computational model can be utilized to compute values from inputs based on a pre-programmed or learned function or method. For example, a neural network may include one or more input neurons that can be activated by inputs. Activations of these neurons can then be passed on, weighted, and transformed by a function to other neurons. Neural networks may range from those with only one layer of single direction logic to multi-layer of multi-directional feedback loops. A neural network can use weights to change the parameters of the network's throughput. A neural network can learn by input from its environment or from self-teaching using written-in rules.

In another example shown in Model B, the disclosed devices, systems, and methods for learning and using AIMS may include a graph or graph-like data structure. As such, machine learning, knowledge representation or structure, pattern recognition, decision making, and/or other artificial intelligence functionalities may include Nodes N (i.e., vertices, points, etc.) and Connections C (i.e., edges, arrows, lines, arcs, etc.) organized as a graph. A graph can be utilized as a predictive modelling approach in machine learning. In general, any Node N in a graph can be connected to any other Node N. A Connection C may include unordered pair of Nodes N in an undirected graph or ordered pair of Nodes N in a directed graph. Nodes N can be part of the graph structure or external entities represented by indices or references. Nodes N, Connections C, and/or operations of a graph may include any features, functionalities, and embodiments of the aforementioned Nodes N, Connections C, and/or operations of a neural network, and vice versa.

In a further example shown in Model C, the disclosed devices, systems, and methods for learning and using AIMs may include a tree or tree-like structure. As such, machine learning, knowledge representation or structure, pattern recognition, decision making, and/or other artificial intelligence functionalities may include Nodes N and Connections C (i.e., references, edges, etc.) organized as a tree. A tree can be utilized as a predictive modelling approach in machine learning. In general, a Node N in a tree can be connected to any number (i.e., including zero, etc.) of children Nodes N (i.e., similar to a tree, etc.). In some aspects, a collection of trees can be utilized where each tree may represent a set of related conversational paths such as, for example, paths concerning a topic or concept. Nodes N, Connections C, and/or operations of a tree may include any features, functionalities, and embodiments of the aforementioned Nodes N, Connections C, and/or operations of a neural network and/or graph, and vice versa.

In a further example shown in Model D, the disclosed devices, systems, and methods for learning and using AIMs may include a sequence or sequence-like structure. As such, machine learning, knowledge representation or structure, pattern recognition, decision making, and/or other artificial intelligence functionalities may include a structure of Nodes N and Connections C organized as a sequence.

In some aspects, Connections C may be optionally omitted from a sequence. A sequence can be utilized as a predictive modelling approach in machine learning. In some aspects, a sequence can be used to store a single data point. In other aspects, a sequence can be used to store multiple concatenated data points. Nodes N, Connections C, and/or operations of a sequence may include any features, functionalities, and embodiments of the aforementioned Nodes N, Connections C, and/or operations of a neural network, graph, and/or tree, and vice versa.

In yet another example the disclosed devices, systems, and methods for learning and using AIMS may include a search-based model and/or technique. As such, machine learning, knowledge representation or structure, pattern recognition, decision making, and/or other artificial intelligence functionalities may include searching through a collection of possible solutions. For example, a search method can search through a neural network, graph, tree, list, or other data structure that includes data elements of interest. A search may use heuristics to limit the search for solutions by eliminating choices that are unlikely to lead to the goal. Heuristic techniques may provide a best guess solution. A search can also include optimization. For example, a search may begin with a guess and then refine the guess incrementally until no more refinements can be made. In a further example, the disclosed devices, systems, and methods for learning and using AIMS may include logic-based model and/or technique. As such, machine learning, knowledge representation or structure, pattern recognition, decision making, and/or other artificial intelligence functionalities can use formal or other type of logic. Logic based models may involve making inferences or deriving conclusions from a set of premises. As such, a logic based system can extend existing knowledge or create new knowledge automatically using inferences. Examples of the types of logic that can be utilized include propositional or sentential logic that comprises logic of statements which can be true or false; first-order logic that allows the use of quantifiers and predicates and that can express facts about objects, their properties, and their relations with each other; fuzzy logic that allows degrees of truth to be represented as a value between O and 1 rather than simply O (false) or 1 (true), which can be used for uncertain reasoning; subjective logic that comprises a type of probabilistic logic that may take uncertainty and belief into account, which can be suitable for modelling and analyzing situations involving uncertainty, incomplete knowledge and different world views; and/or other types of logic

In a further example the disclosed devices, systems, and methods for learning and using AIMs may include a probabilistic model and/or technique. As such, machine learning, knowledge representation or structure, pattern recognition, decision making, and/or other artificial intelligence functionalities can be implemented to operate with incomplete or uncertain information where probabilities may affect outcomes. A Bayesian network, among other models, is an example of a probabilistic tool used for purposes such as reasoning, learning, planning, perception, and/or others. One of ordinary skill in art will understand that the aforementioned artificial intelligence models and/or techniques are described merely as examples of a variety of possible implementations, and that while all possible artificial intelligence models and/or techniques are too voluminous to describe, other artificial intelligence models and/or techniques known in art are within the scope of this disclosure. One of ordinary skill in art will also recognize that an intelligent system may solve a specific problem by using any model and/or technique that works such as, for example, some systems can be symbolic and logical, some can be sub-symbolic neural networks, some can be deterministic or probabilistic, some can be hierarchical, some may include searching techniques, some may include optimization techniques, while others may use other or a combination of models and/or techniques. In general, any artificial intelligence model and/or technique can be utilized that can support AIM functionalities.

Example Computer System

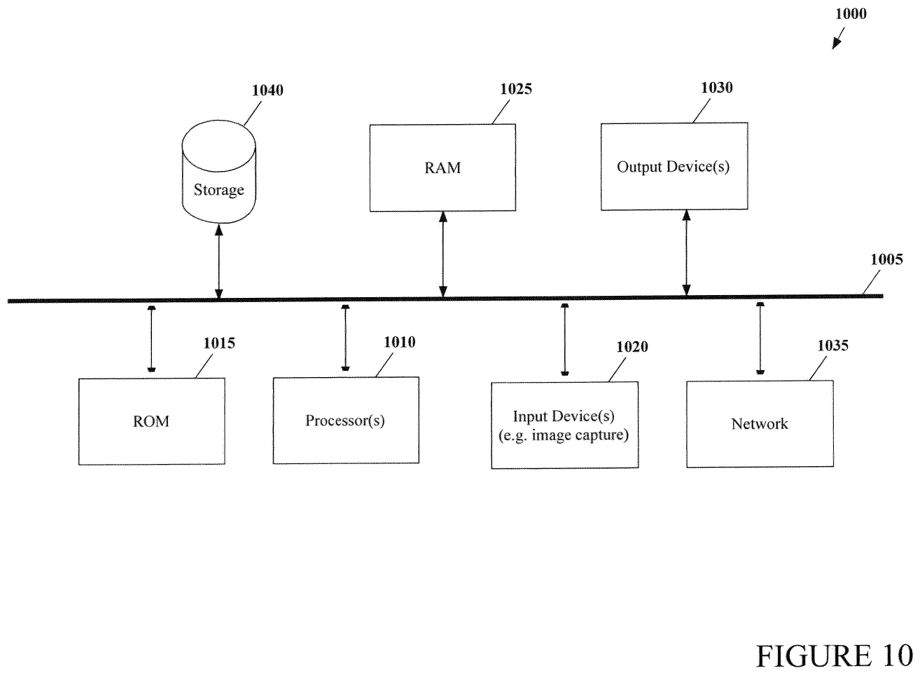

FIG. 10 illustrates an exemplary a system 1000 that may implement the system. The electronic system 1000 of some embodiments may be a mobile apparatus. The electronic system includes various types of machine-readable media and interfaces. The electronic system includes a bus 1005, processor(s) 1010, read only memory (ROM) 1015, input device(s) 1020, random access memory (RAM) 1025, output device(s) 1030, a network component 1035, and a permanent storage device 1040.

The bus 1005 communicatively connects the internal devices and/or components of the electronic system. For instance, the bus 1005 communicatively connects the processor(s) 1010 with the ROM 1015, the RAM 1025, and the permanent storage 1040. The processor(s) 1010 retrieve instructions from the memory units to execute processes of the invention.

The processor(s) 1010 may be implemented with one or more general-purpose and/or special-purpose processors. Examples include microprocessors, microcontrollers, DSP processors, and other circuitry that can execute software. Alternatively, or in addition to the one or more general-purpose and/or special-purpose processors, the processor may be implemented with dedicated hardware such as, by way of example, one or more FPGAs (Field Programmable Gate Array), PLDs (Programmable Logic Device), controllers, state machines, gated logic, discrete hardware components, or any other suitable circuitry, or any combination of circuits.

Many of the above-described features and applications are implemented as software processes of a computer programming product. The processes are specified as a set of instructions recorded on a machine readable storage medium (also referred to as machine readable medium). When these instructions are executed by one or more of the processor(s) 1010, they cause the processor(s) 1010 to perform the actions indicated in the instructions.

Furthermore, software shall be construed broadly to mean instructions, data, or any combination thereof, whether referred to as software, firmware, middleware, microcode, hardware description language, or otherwise. The software may be stored or transmitted over as one or more instructions or code on a machine-readable medium. Machine-readable media include both computer storage media and communication media including any medium that facilitates transfer of a computer program from one place to another. A storage medium may be any available medium that can be accessed by the processor(s) 1010. By way of example, and not limitation, such machine-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a processor. Also, any connection is properly termed a machine-readable medium. For example, if the software is transmitted from a website, server, or other remote source using a coaxial cable, fiber optic cable, twisted pair, digital subscriber line (DSL), or wireless technologies such as infrared (IR), radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk, and Blu-ray® disc where disks usually reproduce data magnetically, while discs reproduce data optically with lasers. Thus, in some aspects machine-readable media may comprise non-transitory machine-readable media (e.g., tangible media). In addition, for other aspects machine-readable media may comprise transitory machine-readable media (e.g., a signal). Combinations of the above should also be included within the scope of machine-readable media.

Also, in some embodiments, multiple software inventions can be implemented as sub-parts of a larger program while remaining distinct software inventions. In some embodiments, multiple software inventions can also be implemented as separate programs. Any combination of separate programs that together implement a software invention described here is within the scope of the invention. In some embodiments, the software programs, when installed to operate on one or more electronic systems 1000, define one or more specific machine implementations that execute and perform the operations of the software programs.

The ROM 1015 stores static instructions needed by the processor(s) 1010 and other components of the electronic system. The ROM may store the instructions necessary for the processor(s) 1010 to execute the processes provided by the system. The permanent storage 1040 is a non-volatile memory that stores instructions and data when the electronic system 1000 is on or off. The permanent storage 1040 is a read/write memory device, such as a hard disk or a flash drive. Storage media may be any available media that can be accessed by a computer. By way of example, the ROM could also be EEPROM, CD-ROM or other optical disk storage, magnetic disk storage or other magnetic storage devices, or any other medium that can be used to carry or store desired program code in the form of instructions or data structures and that can be accessed by a computer.

The RAM 1025 is a volatile read/write memory. The RAM 1025 stores instructions needed by the processor(s) 1010 at runtime, the RAM 1025 may also store the real-time video or still images acquired by the system. The bus 1005 also connects input and output devices 1020 and 1030. The input devices enable the user to communicate information and select commands to the electronic system. The input devices 1020 may be a keypad, image capture apparatus, or a touch screen display capable of receiving touch interactions. The output device(s) 1030 display images generated by the electronic system. The output devices may include printers or display devices such as monitors.

The bus 1005 also couples the electronic system to a network 1035. The electronic system may be part of a local area network (LAN), a wide area network (WAN), the Internet, or an Intranet by using a network interface. The electronic system may also be a mobile apparatus that is connected to a mobile data network supplied by a wireless carrier. Such networks may include 3G, HSPA, EVDO, and/or LTE.

It is understood that the specific order or hierarchy of steps in the processes disclosed is an illustration of exemplary approaches. Based upon design preferences, it is understood that the specific order or hierarchy of steps in the processes may be rearranged. Further, some steps may be combined or omitted. The accompanying method claims present elements of the various steps in a sample order, and are not meant to be limited to the specific order or hierarchy presented.

The various aspects of this disclosure are provided to enable one of ordinary skill in the art to practice the present invention. Various modifications to exemplary embodiments presented throughout this disclosure will be readily apparent to those skilled in the art, and the concepts disclosed herein may be extended to other apparatuses, devices, or processes. Thus, the claims are not intended to be limited to the various aspects of this disclosure, but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 18(f) unless the element is expressly recited using the phrase “means for” or, in the case of a method claim, the element is recited using the phrase “step for.”

Thus, an improved prescription management system has been described.

What is claimed is:

1. A method for providing prescription management service comprising:
in a processing system,
receiving a prescription for a user and transmitting the prescription to a system digital wallet of the user;
using the system digital wallet to select a pharmacy to fill the prescription;
presenting the cost of filling the prescription at the selected pharmacy to the user;
receiving confirmation from the user regarding the cost and a method of delivery of the prescription;
receiving payment for the prescription;
filling the prescription based on the method of delivery.

2. The method of claim 1 wherein the system digital wallet is implemented as an app on a computing device of the user.

3. The method of claim 1 wherein the system digital wallet obtains authorization from an insurance company when prior authorization is required prior to filling the prescription.

4. The method of claim 3 wherein a step of determining if prior authorization is required is performed using artificial intelligence (AI).

5. The method of claim 1 wherein the system digital wallet determines if cash-pay programs are available for the prescription when prior authorization is required and denied.

6. The method of claim 1 wherein the processing system use artificial intelligence (AI) to provide intelligent routing of prescriptions.

7. The method of claim 6 wherein the AI is trained using a reinforcement learning method incorporating a loss function.

8. The method of claim 7 where the system routes the prescription only to pharmacies capable of filling the prescription based on prescription characteristics and user preferences.

9. The method of claim 1 where the system provides text and audio-visual information regarding the prescription to the system digital wallet.

10. The method of claim 1 wherein the system provides drug interaction information to the user based on a historical list of prescriptions of the user stored in conjunction with the digital wallet.
